(19) United States
(12) Patent Application Publication
(10) Pub. No.: US 2014/0106405 A1
(43) Pub. Date: Apr. 17, 2014

(54) MAMMALIAN CELL CULTURE PROCESSES FOR PROTEIN PRODUCTION

(71) Applicant: Bristol-Myers Squibb Company, Princeton, NJ (US)

(72) Inventors: Jun Tian, Manlius, NY (US); Michael Borys, Groton, MA (US); Zhengjian Li, Sudbury, MA (US); Nicholas Abuabsi, Groton, MA (US); Angela Au, Liverpool, NY (US); Nan-xin Qian, Manlius, NY (US); Xiao-ping Dai, Flemington, NJ (US)

(21) Appl. No.: 14/052,987

(22) Filed: Oct. 14, 2013

Related U.S. Application Data
(60) Provisional application No. 61/713,812, filed on Oct. 15, 2012.

(51) Int. Cl. C07K 14/705

(52) U.S. Cl. CPC C07K 14/70575
USPC 435/70.3

(57) ABSTRACT

The present invention describes methods and processes for the production of proteins by animal cell or mammalian cell culture. In one aspect, the methods comprise the growth of cells in a growth factor/protein/peptide free medium. In another aspect, the methods comprise the addition of growth factors during the production phase. The methods sustain a high viability of the cultured cells, and can yield an increased end titer of protein product, and a high quality of protein product.

### FIELD OF THE INVENTION

The present invention relates to new processes for culturing mammalian cells which produce a protein product. Performance of the cell culturing processes result in high cell viability and can also result in high product quality and productivity.

### BACKGROUND OF THE INVENTION

Animal cell culture, notably mammalian cell culture, is preferably used for the expression of recombinantly produced proteins for therapeutic and/or prophylactic applications.

In general, protein expression levels in mammalian cell culture-based systems are considerably lower than in microbial expression systems, for example, bacterial or yeast expression systems. However, bacterial and yeast cells are limited in their ability to optimally express high molecular weight protein products, to properly fold a protein having a complex steric structure, and/or to provide the necessary post-translational modifications to mature an expressed glycoprotein, thereby affecting the immunogenicity and clearance rate of the product.

As a consequence of the limitations of the culturing of animal or mammalian cells, particularly animal or mammalian cells which produce recombinant products, the manipulation of a variety of parameters has been investigated, including the employment of large-scale culture vessels; altering basic culture conditions, such as incubation temperature, dissolved oxygen concentration, pH, and the like; the use of different types of media and additives to the media; and increasing the density of the cultured cells. In addition, process development for mammalian cell culture would benefit from advances in the ability to extend run times to increase final product concentration while maintaining high product quality. Run times of cell culture processes, particularly non-continuous processes, are usually limited by the remaining viability of the cells, which typically declines over the course of the run. The maximum possible extension of high cell viabilities is therefore desired while maintaining product quality. Protein purification concerns offer yet another motivation for minimizing decreases in viable cell density and maintaining high cell viability. The presence of cell debris and the contents of dead cells in the culture can negatively impact on the ability to isolate and/or purify the protein product at the end of the culturing run. By keeping cells viable for a longer period of time in culture, there is thus a concomitant reduction in the contamination of the culture medium by cellular proteins and enzymes that can cause degradation and ultimate reduction in quality of the desired protein produced by the cells.

Various parameters have been investigated to achieve high cell viability in cell cultures. One parameter involved a single lowering of the culture temperature following initial culturing at 37° C. (for example, Roessler et al., Enzyme and Microbial Technology, 18:423-427 (1996); U.S. Pat. Nos. 5,705,364 and 5,721,121 to Etcheverry, T. et al. (1998); U.S. Pat. No. 5,976,833 to Furukawa, K. et al. (1999); U.S. Pat. No. 5,851,800 to Adamson, L. et al.; WO 99/61650 and WO 00/65070 to Genentech, Inc.; WO 00/36092 to Biogen, Inc.; and U.S. Pat. No. 4,357,422 to Girard et al.).

Other parameters investigated involved the addition of components to the culture. The addition of dextran sulfate and polyvinyl sulfate to CHO 111-10PF cell line was found to increase day 3 viable cell density and viability relative to the control culture (Zhangi et al., Biotechnol. Prog., 16:319-325 (2000)). The effect of dextran sulfate or polyvinyl sulfate during the death phase was however not reported. Dextran sulfate and polyvinyl sulfate were also reported to be effective at preventing cell aggregation.

Protein therapeutics are inherently heterogeneous owing to their size, complexity of structure, and the nature of biological production (Chirino et al., Nat. Biotechnol., 22:1383-1391 (2004)). Even in the “pure” protein solution, there will be some percentage of low molecular weight fragments, high molecular weight species, and various degrees of chemical modifications. The formation of high molecular weight species is usually due to protein aggregation, which is a common issue encountered during manufacture of biologics. Typically, the presence of aggregates is considered to be undesirable because of the concern that the aggregates may lead to an immunogenic reaction or may cause adverse events on administration (Cromwell et al., AAPS J., 8:E572-E579 (2006)). Although some types of aggregates of biologics may function normally, it is still important to maintain consistency in product quality since product consistency is a prerequisite for regulatory approval.

Aggregates of proteins may arise from several mechanisms and occur at each stage during the manufacturing process. In cell culture, secreted proteins may be exposed to the conditions that are unfavorable for protein stability; but more often, accumulation of high amounts of protein may lead to intracellular aggregation owing to either the interactions of unfolded protein molecules or to inefficient recognition of the nascent peptide chain by molecular chaperones responsible for proper folding (Cromwell et al., AAPS J., 8:E572-E579 (2006)). In the endoplasmic reticulum (ER) of cells, disulfide bond of newly synthesized protein is formed in an oxidative environment. Under normal condition, protein sulfhydryls are reversibly oxidized to protein disulfides and sulfenic acids, but the more highly oxidized states such as the sulfinic and sulfonic acid forms of protein cysteines are irreversible (Thomas et al., Exp. Gerontol., 36:1519-1526 (2001)). Hyper-oxidized proteins may contain incorrect disulfide bonds or have mixed disulfide bonds with other luminal ER proteins; in either case it leads to protein improper folding and aggregation. It is therefore crucial to maintain a properly controlled oxidative environment in the ER. In this regard, Cuozzo et al. (Nat. Cell Biol., 1:130-135 (1990)) initially demonstrated that in yeasts glutathione buffered against ER hyperoxidation and later on Chakravarthi et al. (J. Biol. Chem., 279:39872-39879 (2004)) confirmed that in mammalian cells glutathione was also required to regulate the formation of native disulfide bonds within proteins entering the secretory pathway.

With increasing product concentration in the culture, it can be observed in cell culture processes that the product quality decreases. High abundance of a protein produced by cells in culture is optimally accompanied by high quality of the protein that is ultimately recovered for an intended use.

Recombinantly produced protein products are increasingly becoming medically and clinically important for use as therapeutics, treatments and prophylactics. Therefore, the development of reliable cell culture processes that economically and efficiently achieve an increased final protein product concentration, in conjunction with a high level of product quality, fulfills both a desired and needed goal in the art.

### SUMMARY OF THE INVENTION

The present invention provides new processes for the production of proteins by animal or mammalian cell cultures. These new processes achieve increased viable cell density, cell viability, productivity and decreased protein aggregation.

One aspect of the invention concerns the growth of cells in a growth factor/protein/peptide free media throughout the culture process. In this aspect, cell culture processes of this invention can advantageously achieve an enhanced specific productivity of the protein produced by the cultured cells. More specifically, in accordance with this invention, growth factor/protein/peptide free media utilized during the cell culturing period sustains a high cell viability of the cells in the culture and can provide a high quantity and quality of produced product throughout an entire culture run. Also, according to one aspect of the invention, growth factor/protein/peptide free media utilized during the culturing processes can advantageously allow for an extension of the production phase of the culture. During the extended production phase, the titer of the desired product is increased; the product quality is maintained at a high level; protein aggregation level is maintained at lower level and cell viability is also maintained at a high level. In addition, the extended production phase associated with the culturing processes of the invention allows for the production of product beyond that which is produced during a standard production phase.

Another aspect of this invention concerns the addition of one or more growth factors to the growth factor-free cell culture after inoculation. In accordance with this invention, addition of one or more growth factors after inoculation sustains a high cell viability of the cells in the culture and can provide a high quantity and quality of produced product throughout an entire culture run. During the production phase, the titer of the desired product is increased; the product quality is maintained at a high level; protein aggregation level is maintained at lower level and cell viability is also maintained at a high level.

In one particular aspect, the present invention provides a process (or method) in which the specific productivity is enhanced, the protein aggregation level was reduced by the addition of insulin and/or IGF in the fed medium. In accordance with this particular aspect, the addition of insulin and or IGF sustains a high cell viability of the culture, thereby enabling an extended production phase during which the titer of product, preferably recombinant product is increased and the product quality is maintained at high level.

In one aspect of this invention, one or more growth factors are added to a culture at the time of inoculation or at a time after inoculation that is before the beginning of the initial death phase, or is during the initial growth phase, or is during the second half of the initial growth phase, or is on or about the end of the initial growth phase. In accordance with this aspect of the invention, the growth phase is extended and/or the onset of the death phase is delayed for a period of time, such as several days.

Further aspects, features and advantages of the present invention will be appreciated upon a reading of the detailed description of the invention and a consideration of the drawings/figures.

### DESCRIPTION OF THE DRAWINGS/FIGURES

FIG. 1 shows viable cell density (VCD), percent viability and doubling time for clone 40A6. The CHO cell line used in this study was originally subcloned from DG44 parental cells and cultured in a growth factor/protein/peptide free basal medium (GF-free) or basal medium with the growth factor (GF) insulin, as described in Example 1.

FIG. 2 shows viable cell density (VCD), percent viability and doubling time for clone 63C2. The CHO cell line used in this study was originally subcloned from DG44 parental cells and cultured in a growth factor/protein/peptide free basal medium (GF-free) or basal medium with the growth factor (GF) insulin, as described in Example 1.

FIG. 3 shows the change in expression/phosphorylation of proteins from the mTOR pathway when clone 40A6 cells are grown with (INS) and without insulin (INS-F), as described in Example 2.

FIG. 4 shows viable cell density (VCD), percent viability, aCD40L protein titer, percent aCD40L monomer for clone 40A6 grown in basal medium with 1 mg/L insulin and 10 mg/L in the feed medium (INS) or without insulin in the basal or feed medium (INS-Free) as described in Example 3.

FIG. 5 shows viable cell density (VCD), percent viability, aCD40L protein titer for clone 40A6 grown in basal medium with 1 mg/L insulin and 10 mg/L in the feed medium (INS) or without insulin in the basal or feed medium (INS Free). Samples were from fed-batch cultures with cells at passage 26 (p26), passage 41 (p41), passage 15 (p15), passage 18 (p18) and passage 33 (p33) as described in Example 3.

FIGS. 6 and 6A show viable cell density (VCD), percent viability, aCD40L protein titer, percent aCD40L high molecular weight (HMW) for clone 63C2 grown in a 5 L reactor where insulin was added back to the basal and feed media after inoculation as described in Example 4.

FIGS. 7 and 7A show viable cell density (VCD), percent viability, aCD40L protein titer, percent aCD40L high molecular weight (HMW) for clone 63C2 grown in a 5 L reactor where the growth factors insulin (INS) and/or LONG®R3 (LR3) are added back to the basal and feed media after inoculation as described in Example 5.

### DETAILED DESCRIPTION OF THE INVENTION

The present invention describes new processes for the production of proteins, preferably recombinant protein products, in mammalian or animal cell culture. These processes achieve increased viable cell density, cell viability, productivity and decreased protein aggregation.

Chinese hamster ovary (CHO) cells have been one of the major cell types used for the production of recombinant therapeutics. For the large-scale manufacturing of such therapeutics in CHO cells with chemically defined cell culture media, growth factors (GF) are widely used to promote cell growth and productivity. However, the use of growth factors not only significantly increases the cost of manufacturing, but potentially also impacts the process performance and robustness due to complicated cell signaling from the growth factors.

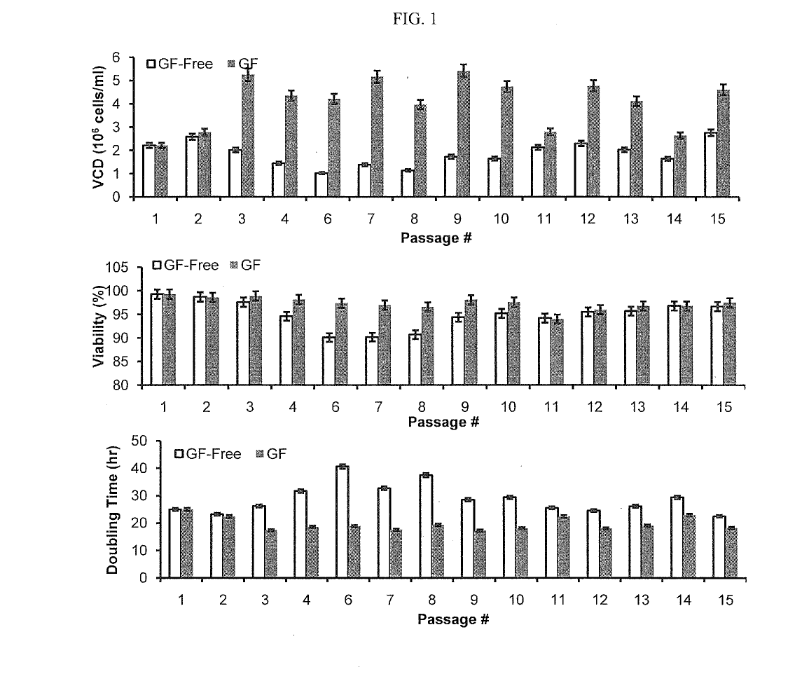

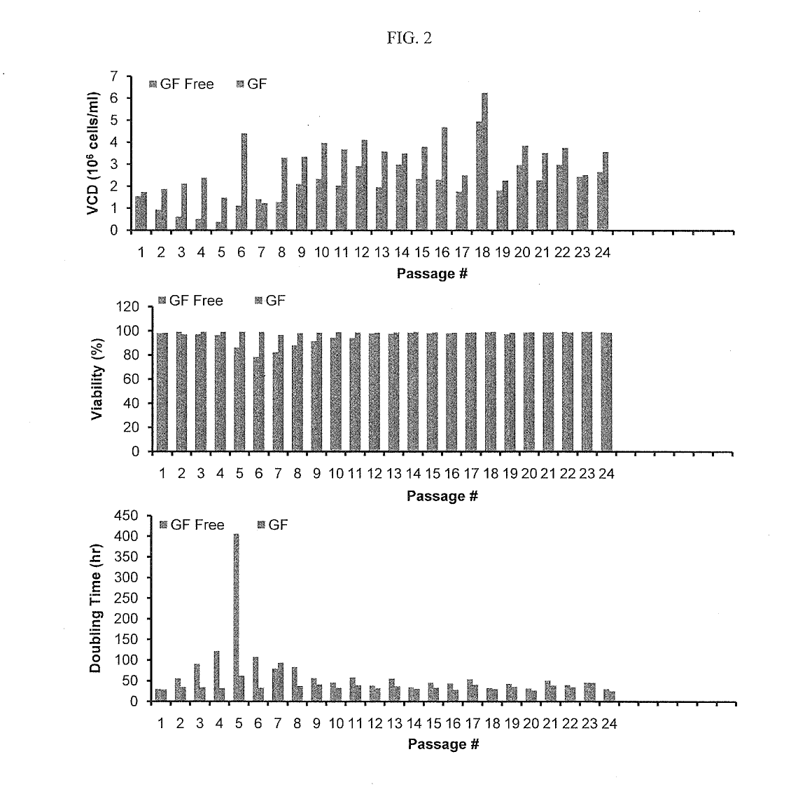

One embodiment of the invention shows that signaling from growth factors could be removed for CHO cell culture. Example 1 shows that two different dihydrofolate reductase (DHFR) clones expressing a domain fusion antibody, can be adapted to growth factor free (GF-free) media by continuously culturing them in the media for up to nine passages(see FIGS. 1 and 2). Spent medium analysis revealed that cells under the GF-free condition elevated the consumption of amino acids up to two-fold compared to the growth factor condition, suggesting that a shift in cellular metabolism may be required to adapt to GF-free culture.

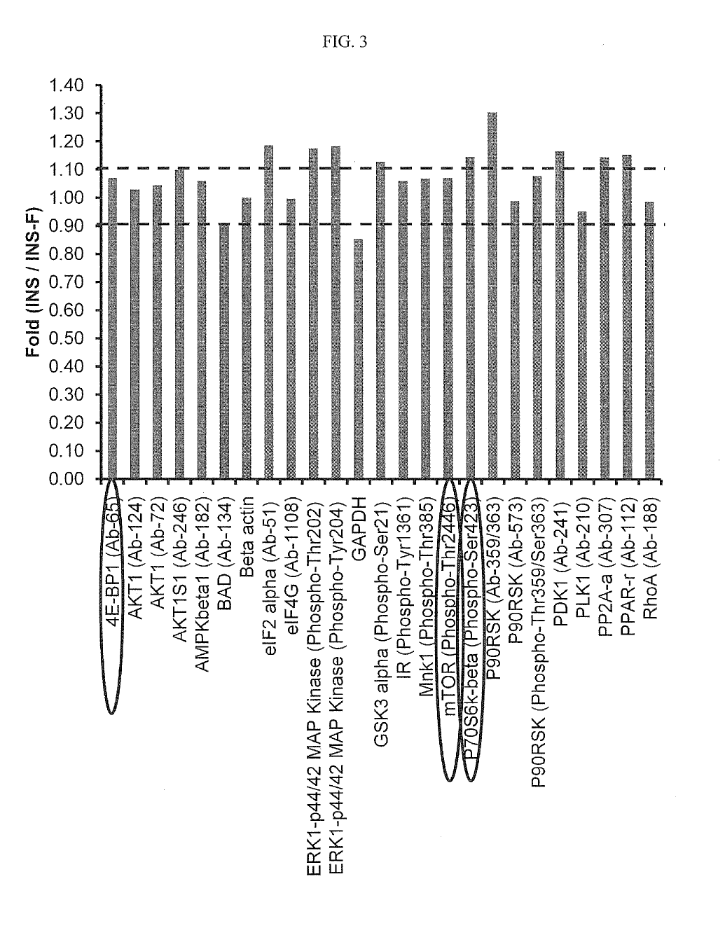

Interestingly, as shown in Example 2, the mTOR pathway, which regulates cell growth and protein production, was not significantly impacted by growth factor removal as shown by antibody arrays that target 138 proteins involved in the mTOR pathway (see FIG. 3).

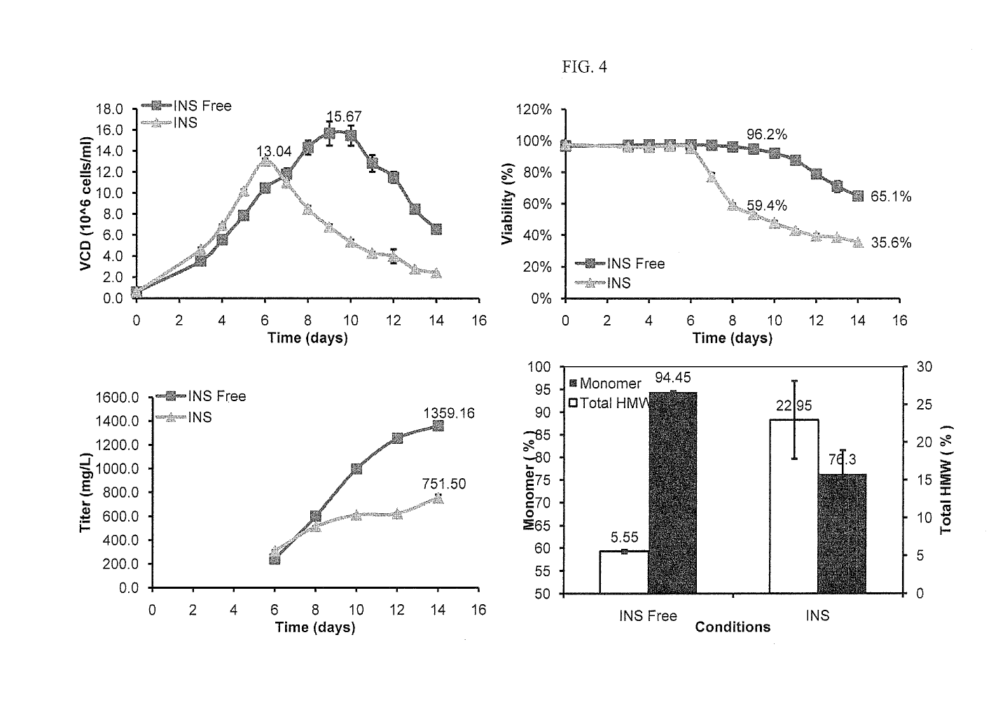

Production assays of fed-batch cultures described in Example 3 found that the GF-free condition increased peak viable cell density to 15.7×10⁶ cells/ml from 13.0×10⁶ cells/ml for the GF condition, and improved the cell viability at the later stages of the culture, from 59.4% for the GF condition to 96.2% on day 8, and from 35.6% to 65.1% on day 14. As a result, the protein titer was increased by 80.9%. In addition, the protein quality was enhanced under the GF-free condition as the high molecular species decreased to 4.8% from 14.3% for the GF condition (see FIG. 4).

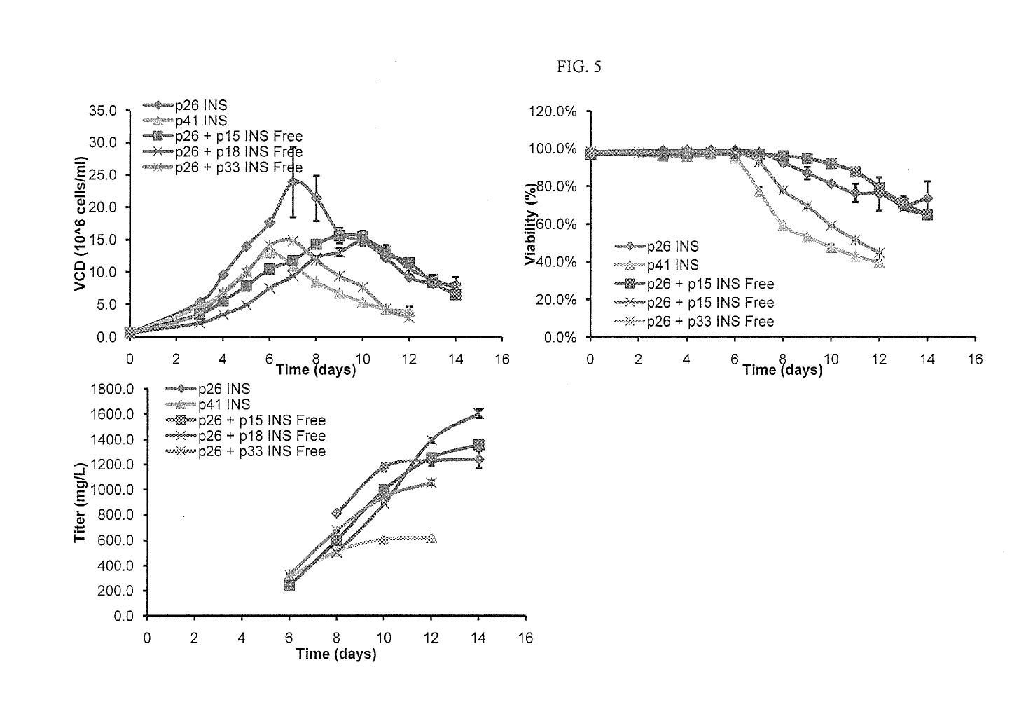

Additionally, FIG. 5 shows that the stability of the cells was improved under the GF-free condition. The productivity decreased by 50.0% after cells were cultured under the GF condition for 15 passages (7 weeks), while cells cultured under GF-free condition maintained their productivity.

Overall, our results demonstrate that chemically defined medium free of proteins and peptides is capable of propagating CHO cells, delivering productivities comparable to the GF condition, and better maintaining the stability of clones.

In one embodiment, the invention is directed to a cell culturing process comprising: culturing host/inoculum cells, which express a protein of interest, in growth factor/protein/peptide free media under conditions for protein production.

In other embodiments, the invention is directed to a cell culturing process for increasing cell viability at later stages of culture comprising culturing host cells, which express a protein of interest; in growth factor/protein/peptide free media, wherein the end stage cell viability is increased compared to end stage cell viability in cells grown in media comprising growth factor/protein/peptide.

In another embodiment, the invention is directed to a cell culturing process for increasing production of a protein of interest comprising culturing host cells, which express a protein of interest; in growth factor/protein/peptide free media, wherein the protein titer is increased compared to protein titer in cells grown in media comprising growth factor/protein/peptide.

In another embodiment, the invention is directed to a cell culturing process for reducing the percentage of protein aggregation comprising: culturing host cells, which express a protein of interest; in growth factor/protein/peptide free media, wherein the percentage of high molecular weight species is decreased compared to the percentage of high molecular weight species in cells grown in media comprising growth factor/protein/peptide.

A growth factor is a naturally occurring substance capable of stimulating cellular growth, and cellular differentiation. Usually it is a protein or a steroid hormone. Growth factors are important for regulating a variety of cellular processes. Growth factors typically act as signaling molecules between cells. They often promote cell differentiation and maturation, which varies between growth factors.

In one embodiment, the invention is directed to a cell culturing process comprising: culturing host/inoculum cells, which express a protein of interest, in growth factor/protein/peptide free media; and adding one or more growth factors to the production cell culture.

Growth factors useful in the cell culture process of the invention include, but are not limited to, insulin (GIBCO® rHu AOF Insulin, Biocon), platelet-derived growth factor (PDGF), basic fibroblast growth factor (bFGF), epidermal growth factor (EGF) (LONG®EGF, RepliGen Bioprocessing), insulin-like growth factor (IGF) (LONG®R3IGF-1, RepliGen Bioprocessing), transforming growth factor alpha (TGF-α) (LONG®TGF-α, RepliGen Bioprocessing), erythropoietin, steroids, serum, nerve growth factor (NGF), fibroblast growth factor (FGF) and colony-stimulating factor (CSF). The compounds are readily available from the listed sources, or readily obtainable through means known to one of skill in the art.

In one embodiment the growth factors include but are not limited to insulin and/or insulin-like growth factor (IGF).

In one embodiment of the invention, growth factor is added at inoculation or may be a component of the basal medium. Inoculation takes place on day 0.

In one embodiment of the invention, growth factor is added at a time after inoculation, i.e., it is not present in the basal medium and not present at inoculation. In one specific embodiment, growth factor is added on day 1 of the culture or later.

In accordance with the invention, growth factor may be added to the cell culture one time, two times, three times, or any number of times during the specified time period. One or more growth factors may be used in conjunction. That is, any given single addition of a growth factor may include the addition of one or more other growth factors. Similarly, if there is more than one addition of a growth factor, different growth factors may be added at the different additions. Additional compounds and substances, including growth factor, may be added to the culture before, with or after the addition of growth factor—either during or not during the specified time period. In a specific embodiment, growth factor is added with the basal medium after inoculation. In another embodiment, growth factor is added with the feed medium. In a specific embodiment, growth factor is added with the basal medium after inoculation and with the feed medium. In another specific embodiment, one growth factor is added.

In accordance with the invention, growth factor may be added to the cell culture by any means. Means of adding growth factor include, but are not limited to, dissolved in water, dissolved in an acid, dissolved in basal medium, dissolved in feed medium, dissolved in a suitable medium, in the form in which it is obtained or any combination thereof.

In one embodiment of the invention, insulin is added as a solution where the insulin is dissolved in 1 M HCL that is then diluted with water for further use (i.e., such as adding insulin to the feed medium).

In one embodiment of the invention, growth factor is added to bring the concentration in the culture to an appropriate level. As non-limiting examples, insulin is added to a concentration of 1 μg/L-10 mg/L. In another embodiment of the invention, insulin is added to a concentration of 4 μg/L-10 mg/L. In still another embodiment of the invention insulin is added to a concentration of 40 μg/L-10 mg/L.

In one embodiment of the invention, growth factor is added to the basal medium in an amount of about 1 μg/L to 10 mg/L. In another embodiment of the invention, growth factor is added to the basal medium in an amount of about 1 μg/L to 1 mg/L.

In one embodiment of the invention, growth factor is added to the feed medium in an amount of about 1 μg/L to 10 mg/L. In another embodiment of the invention, growth factor is added to the feed medium in an amount that is from 5 fold to 10 fold that of the basal medium. In a non-limiting example, insulin may be added to the feed medium in an amount of about 10 mg/L and/or insulin-like growth factor may be added to the feed medium in an amount of about 40 μg/L.

In accordance with the invention, the culture may be run for any length of time after addition of growth factor. The culture run time may be determined by one of skill in the art, based on relevant factors such as the quantity and quality of recoverable protein, and the level of contaminating cellular species (e.g., proteins and DNA) in the supernatant resulting from cell lysis, which will complicate recovery of the protein of interest.

In particular embodiments of the cell culturing process and method of increasing cell viability of the invention, growth factor is added at a time after inoculation that is before the beginning of the initial death phase. Alternatively, growth factor is added at a time after inoculation that is during the initial growth phase, or insulin is added during the second half the initial growth phase, or growth factor is added on or about the end of the initial growth phase.

The initial growth phase refers to the growth phase that is observed in the absence of the specified addition of growth factor. The initial death phase refers to the death phase that is observed in the absence of the specified addition of growth factor.

The initial growth phase may end when the initial death phase begins, or there may be a stationary phase of any length between the initial growth phase and the initial death phase.

For example, in a cell culture in which the initial growth phase is from day 0 to day 6 and the initial death phase begins on day 7, in a particular embodiment growth factor is added at a time after inoculation and before day 7. In a specific embodiment, growth factor is added after inoculation and by day 6. In a specific embodiment, growth factor is added between days 1 and 6. In another specific embodiment, growth factor is added with the feed medium on days 3-6. In other specific embodiments, growth factor is added on about day 2, or on day 2.

It has been found (see FIG. 4) that when carrying the present invention the viability of the cell culture is prolonged. A condition, such as addition of growth factor, causes prolonged cell viability if cell viability in the culture is higher for a period of time in the presence of the condition than in the absence of the condition.

Thus, in other embodiments, the invention is directed to (1) a cell culturing process, and (2) a method of prolonging cell viability in a culture comprising: culturing host cells, which express a protein of interest, in growth factor/protein/peptide free media; and adding growth factor to the cell culture; wherein the cell viability of the cell culture is prolonged.

Run times of cell culture processes, particularly non-continuous processes, are usually limited by the remaining viable cell density, which decreases during the death phase. Longer run times may allow higher product titers to be achieved. Product quality concerns also offer a motivation for reducing death rate as the presence of cell debris and the contents of dead cells in the culture can negatively impact on the ability to isolate and/or purify the protein product at the end of the culturing run.

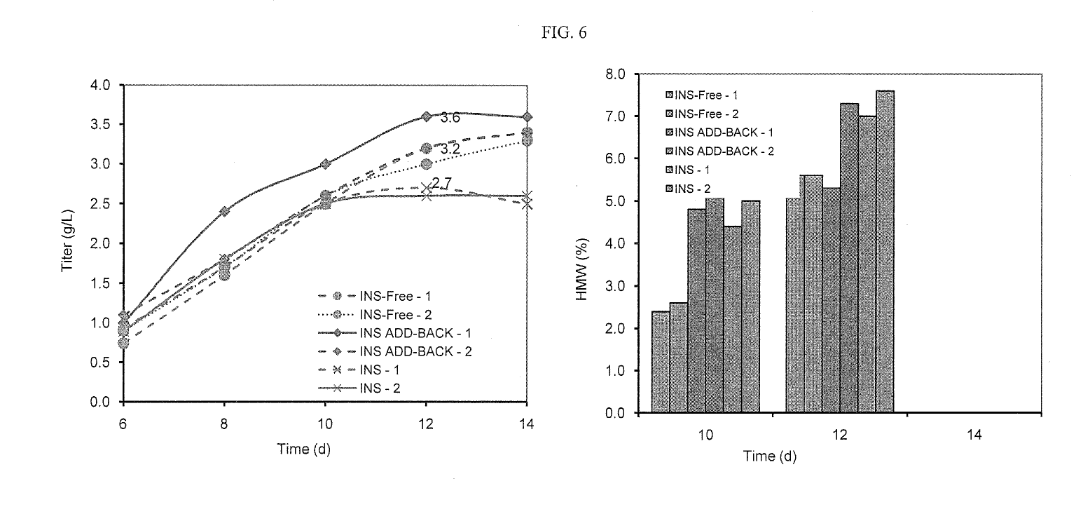

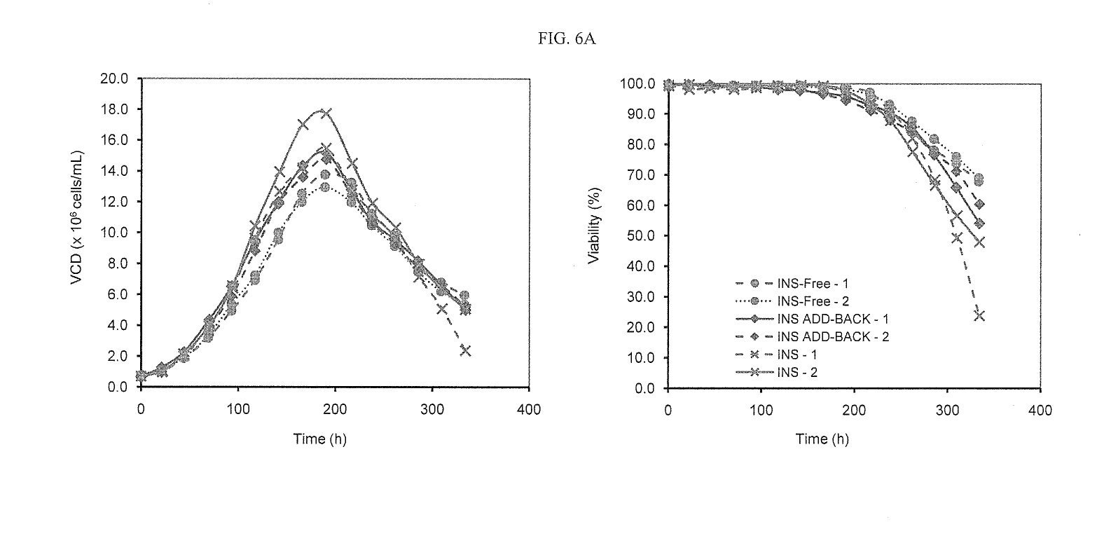

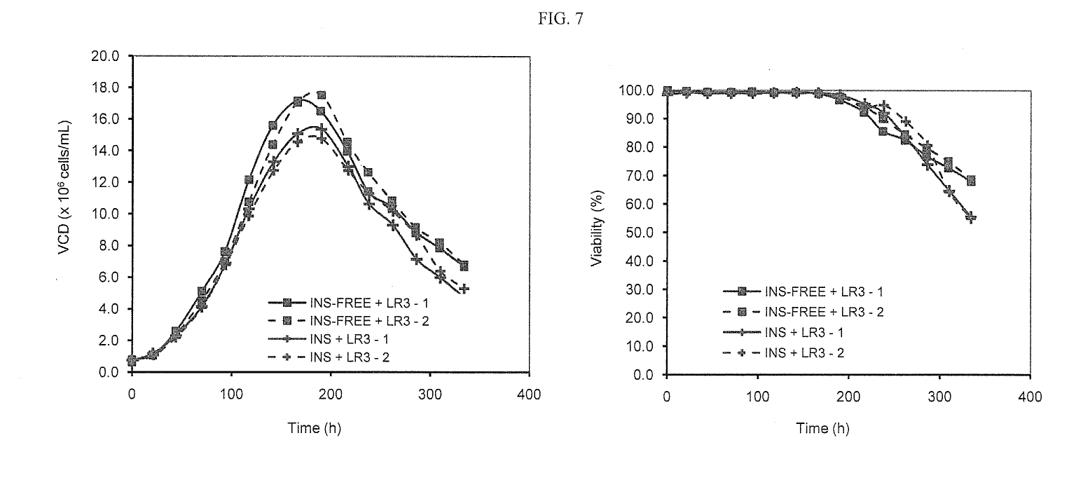

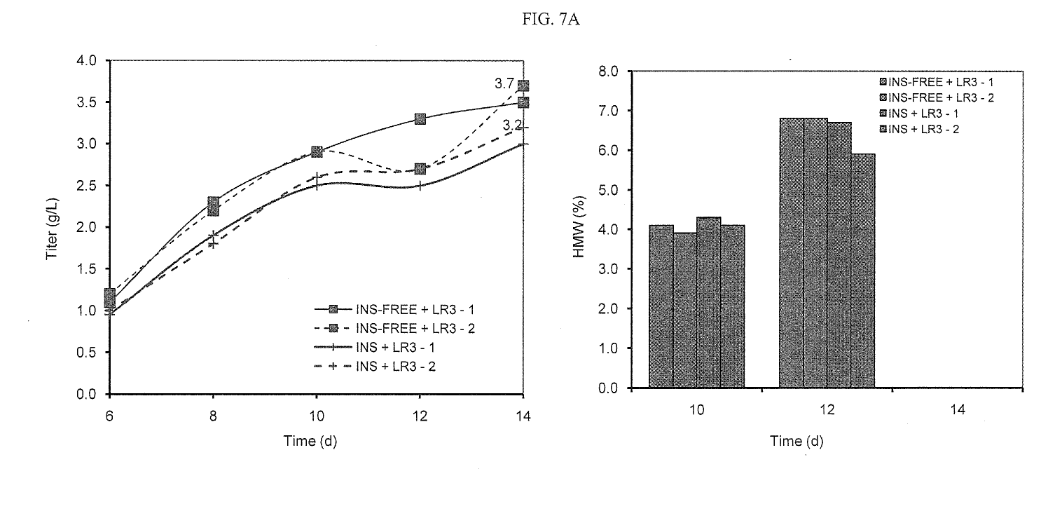

It has been found (see FIG. 6), that addition of growth factor to the cell culture reduces the aggregation of the proteins of interest.

Thus, in other embodiments, the invention is directed to a cell culturing process for reducing the percentage of protein aggregation comprising: culturing host cells, which express a protein of interest; in growth factor/protein/peptide free media and adding growth factor to the cell culture; wherein the percentage of high molecular weight species is decreased.

### Techniques and Procedures Relating to Protein Purification and Analysis

In the culturing methods encompassed by the present invention, the protein produced by the cells is typically collected, recovered, isolated, and/or purified, or substantially purified, as desired, at the end of the total cell culture period using isolation and purification methods as known and practiced in the art. Preferably, protein that is secreted from the cultured cells is isolated from the culture medium or supernatant; however, protein can also be recovered from the host cells, e.g., cell lysates, using methods that are known

Illustratively, for protein recovery, isolation and/or purification, the cell culture medium or cell lysate is centrifuged to remove particulate cells and cell debris. The desired polypeptide product is isolated or purified away from contaminating soluble proteins and polypeptides by suitable purification techniques. The following procedures provide exemplary, yet nonlimiting purification methods for proteins: separation or fractionation on immunoaffinity or ion-exchange columns; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., SEPHADEX® G-75, SEPHAROSE®; protein A SEPHAROSE® chromatography for removal of immunoglobulin contaminants; and the like. Other additives, such as protease inhibitors (e.g., PMSF or proteinase K) can be used to inhibit proteolytic degradation during purification. It will be understood by the skilled practitioner that purification methods for a given polypeptide of interest may require modifications which allow for changes in the polypeptide expressed recombinantly in cell culture.

### Cells, Proteins and Cell Cultures

In the cell culture processes or methods of this invention, the cells can be maintained in a variety of cell culture media. i.e., basal culture media, as conventionally known in the art. For example, the methods are applicable for use with large volumes of cells maintained in cell culture medium, which can be supplemented with nutrients and the like. Typically, “chemically defined cell culturing medium” (also called “chemically defined medium”) is a term that is understood by the practitioner in the art and is known to refer to a nutrient solution in which cells, preferably animal or mammalian cells, are grown and which generally provides at least one or more components from the following: an energy source (usually in the form of a carbohydrate such as glucose); all essential amino acids, and generally the twenty basic amino acids, plus cysteine; vitamins and/or other organic compounds typically required at low concentrations; lipids or free fatty acids, e.g., linoleic acid; and trace elements, e.g., inorganic compounds or naturally occurring elements that are typically required at very low concentrations, usually in the micromolar range. Cell culture medium can also be supplemented to contain a variety of optional components, such as salts, e.g., calcium, magnesium and phosphate, and buffers, e.g., HEPES; nucleosides and bases, e.g., adenosine, thymidine, hypoxanthine; antibiotics, e.g., gentamycin; and cell protective agents, e.g., a PLURONIC® polyol (PLURONIC® F68).

One embodiment of the invention is a cell culture process utilizing a chemically defined cell culture media that is free of growth factors, proteins and peptides, e.g., insulin, transferrin, epidermal growth factor, serum, hydrolyzed protein, hormones, bFGF, IGF, PDGF and free of products or ingredients of animal origin.

As is appreciated by the practitioner, animal or mammalian cells are cultured in a medium suitable for the particular cells being cultured and which can be determined by the person of skill in the art without undue experimentation. Commercially available media can be utilized and include, for example, Minimal Essential Medium (MEM, Sigma, St. Louis, Mo.); Ham's F10 Medium (Sigma); Dulbecco's Modified Eagles Medium (DMEM, Sigma); RPMI-1640 Medium (Sigma); HYCLONE® cell culture medium (HyClone, Logan, Utah); and chemically-defined (CD) media, which are formulated for particular cell types, e.g., CD-CHO Medium (Invitrogen, Carlsbad, Calif.). EX-CELL® CD-CHO (SAFC, Saint Louis).

To the foregoing exemplary media can be added the above-described supplementary components or ingredients, including optional components, in appropriate concentrations or amounts, as necessary or desired, and as would be known and practiced by those having in the art using routine skill.

In addition, cell culture conditions suitable for the methods of the present invention are those that are typically employed and known for batch, fed-batch, or continuous culturing of cells, with attention paid to pH, e.g., about 6.5 to about 7.5; dissolved oxygen (O₂), e.g., between about 5-90% of air saturation and carbon dioxide (CO₂), agitation and humidity, in addition to temperature. As an illustrative, yet nonlimiting, example, a suitable cell culturing medium for the fed-batch processes of the present invention comprises a modified CD-CHO Medium (Invitrogen, Carlsbad, Calif.). A feeding medium can also be employed, such as modified eRDF medium (Invitrogen, Carlsbad, Calif.). One embodiment of the invention utilizes a feeding medium also containing a growth factor, e.g., insulin.

Animal cells, mammalian cells, cultured cells, animal or mammalian host cells, host cells, recombinant cells, recombinant host cells, and the like, are all terms for the cells that can be cultured according to the processes of this invention. Such cells are typically cell lines obtained or derived from mammals and are able to grow and survive when placed in either monolayer culture or suspension culture in medium containing appropriate nutrients.

Numerous types of cells can be cultured according to the methods of the present invention. The cells are typically animal or mammalian cells that can express and secrete, or that can be molecularly engineered to express and secrete, large quantities of a particular protein of interest, into the culture medium. It will be understood that the protein produced by a host cell can be endogenous or homologous to the host cell. Alternatively, the protein is heterologous, i.e., foreign, to the host cell, for example, a human protein produced and secreted by a Chinese hamster ovary (CHO) host cell. In one embodiment, mammalian proteins, i.e., those originally obtained or derived from a mammalian organism, are attained by the methods the present invention and are preferably secreted by the cells into the culture medium.

Proteins of interest may include fusion proteins and polypeptides, chimeric proteins and polypeptides, as well as fragments or portions, or mutants, variants, or analogues of any of the aforementioned proteins and polypeptides are also included among the suitable proteins, polypeptides and peptides that can be produced by the methods of the present invention.

Nonlimiting examples of animal or mammalian host cells suitable for harboring, expressing, and producing proteins for subsequent isolation and/or purification include Chinese hamster ovary cells (CHO), such as CHO-K1 (ATCC CCL-61), DG44 (Chasin et al., Som. Cell Molec. Genet., 12:555-556 (1986); and Kolkekar et al., Biochemistry, 36:10901-10909 (1997)), CHO-K1 Tet-On cell line (Clontech), CHO designated ECACC 85050302 (CAMR, Salisbury, Wiltshire, UK), CHO clone 13 (GEIMG, Genova, IT), CHO clone B (GEIMG, Genova, IT), CHO-K1/SF designated ECACC 93061607 (CAMR, Salisbury, Wiltshire, UK), RR-CHOK1 designated ECACC 92052129 (CAMR, Salisbury, Wiltshire, UK), dihydrofolate reductase negative CHO cells (CHO/-DHFR, Urlaub et al., Proc. Natl. Acad. Sci. USA, 77:4216 (1980)), dp12.CHO cells (U.S. Pat. No. 5,721,121) and FUT8 (alpha-1,6-fucosyltransferase) knockout CHO cell line, Ms704 (Naoko Yamane-Ohnuki et al. Published online 6 Aug. 2004 in Wiley InterScience (www.interscience.wiley.com). DOI: 10.1002/bit.20151); monkey kidney CV1 cells transformed by SV40 (COS cells, COS-7, ATCC® CRL-1651); human embryonic kidney cells (e.g., 293 cells, or 293 cells subcloned for growth in suspension culture, Graham et al., J. Gen. Virol., 36:59 (1977)); baby hamster kidney cells (BHK, ATCC® CCL-10); monkey kidney cells (CV1, ATCC® CCL-70); African green monkey kidney cells (VERO-76, ATCC® CRL-1587; VERO, ATCC® CCL-81); mouse sertoli cells (TM4, Mather, Biol. Reprod., 23:243-251 (1980)); human cervical carcinoma cells (HELA, ATCC® CCL-2); canine kidney cells (MDCK, ATCC® CCL-34); human lung cells (W138, ATCC® CCL-75); human hepatoma cells (HEP-G2, HB 8065); mouse mammary tumor cells (MMT 060562, ATCC® CCL-51); buffalo rat liver cells (BRL 3A, ATCC® CRL-1442); TRI cells (Mather, Ann. NY Acad. Sci., 383:44-68 (1982)); MCR 5 cells; FS4 cells. In one embodiment of the invention the cells are CHO cells, such as CHO/-DHFR cells and CHO/-GS cells.

The cells suitable for culturing in the methods and processes of the present invention can contain introduced, e.g., via transformation, transfection, infection, or injection, expression vectors (constructs), such as plasmids and the like, that harbor coding sequences, or portions thereof, encoding the proteins for expression and production in the culturing process. Such expression vectors contain the necessary elements for the transcription and translation of the inserted coding sequence. Methods which are well known to and practiced by those skilled in the art can be used to construct expression vectors containing sequences encoding the produced proteins and polypeptides, as well as the appropriate transcriptional and translational control elements. These methods include in vitro recombinant DNA techniques, synthetic techniques, and in vivo genetic recombination. Such techniques are described in Sambrook, J. et al., Molecular Cloning, A Laboratory Manual, Cold Spring Harbor Press, Plainview, N.Y. (1989) and in Ausubel, F. M. et al., Current Protocols in Molecular Biology, John Wiley & Sons, New York, N.Y. (1989).

Control elements, or regulatory sequences, are those non-translated regions of the vector, e.g., enhancers, promoters, 5′ and 3′ untranslated regions, that interact with host cellular proteins to carry out transcription and translation. Such elements can vary in their strength and specificity. Depending on the vector system and host cell utilized, any number of suitable transcription and translation elements, including constitutive and inducible promoters, can be used. In mammalian cell systems, promoters from mammalian genes or from mammalian viruses are preferred. The constructs for use in protein expression systems are designed to contain at least one promoter, an enhancer sequence (optional, for mammalian expression systems), and other sequences as necessary or required for proper transcription and regulation of gene expression (e.g., transcriptional initiation and termination sequences, origin of replication sites, polyadenylation sequences).

As will be appreciated by those skilled in the art, the selection of the appropriate vector, e.g., plasmid, components for proper transcription, expression, and isolation of proteins produced in eukaryotic (e.g., mammalian) expression systems is known and routinely determined and practiced by those having skill in the art. The expression of proteins by the cells cultured in accordance with the methods of this invention can placed under the control of promoters such as viral promoters, e.g., cytomegalovirus (CMV), Rous sarcoma virus (RSV), phosphoglycerol kinase (PGK), thymidine kinase (TK), or the α-actin promoter. Further, regulated promoters confer inducibility by particular compounds or molecules. Also, tissue-specific promoters or regulatory elements can be used (Swift, G. et al., Cell, 38:639-646 (1984)), if necessary or desired.

Expression constructs can be introduced into cells by a variety of gene transfer methods known to those skilled in the art, for example, conventional gene transfection methods, such as calcium phosphate co-precipitation, liposomal transfection, microinjection, electroporation, and infection or viral transduction. The choice of the method is within the competence of the skilled practitioner in the art. It will be apparent to those skilled in the art that one or more constructs carrying DNA sequences for expression in cells can be transfected into the cells such that expression products are subsequently produced in and/or obtained from the cells.

In a particular aspect, mammalian expression systems containing appropriate control and regulatory sequences are preferred for use in protein expressing mammalian cells of the present invention. Commonly used eukaryotic control sequences for use in mammalian expression vectors include promoters and control sequences compatible with mammalian cells such as, for example, the cytomegalovirus (CMV) promoter (CDM8 vector) and avian sarcoma virus (ASV), (πLN). Other commonly used promoters include the early and late promoters from Simian Virus 40 (SV40) (Fiers et al., Nature, 273:113 (1973)), or other viral promoters such as those derived from polyoma, Adenovirus 2, and bovine papilloma virus. An inducible promoter, such as hMTII (Karin et al., Nature, 299:797-802 (1982)) can also be used.

Examples of expression vectors suitable for eukaryotic host cells include, but are not limited to, vectors for mammalian host cells (e.g., BPV-1, pHyg, pRSV, pSV2, pTK2 (Maniatis); pIRES (Clontech); pRc/CMV2, pRc/RSV, pSFV1 (Life Technologies); pVPakc Vectors, pCMV vectors, pSG5 vectors (Stratagene), retroviral vectors (e.g., pFB vectors (Stratagene)), pcDNA-3 (Invitrogen), adenoviral vectors; Adeno-associated virus vectors, baculovirus vectors, yeast vectors (e.g., pESC vectors (Stratagene)), or modified forms of any of the foregoing. Vectors can also contain enhancer sequences upstream or downstream of promoter region sequences for optimizing gene expression.

A selectable marker can also be used in a recombinant vector (e.g., a plasmid) to confer resistance to the cells harboring (preferably, having stably integrated) the vector to allow their selection in appropriate selection medium. A number of selection systems can be used, including but not limited to, the Herpes Simplex Virus thymidine kinase (HSV TK), (Wigler et al., Cell, 11:223 (1977)), hypoxanthine-guanine phosphoribosyltransferase (HGPRT), (Szybalska et al., Proc. Natl. Acad. Sci. USA, 48:202 (1992)), and adenine phosphoribosyltransferase (Lowy et al., Cell, 22:817 (1980)) genes, which can be employed in tk-, hgprt-, or aprt-cells (APRT), respectively.

Anti-metabolite resistance can also be used as the basis of selection for the following nonlimiting examples of marker genes: dhfr, which confers resistance to methotrexate (Wigler et al., Proc. Natl. Acad. Sci. USA, 77:357 (1980); and O'Hare et al., Proc. Natl. Acad. Sci. USA, 78:1527 (1981)); glutamine synthase (GS), which confers resistance to methionine sulphoximine; gpt, which confers resistance to mycophenolic acid (Mulligan et al., Proc. Natl. Acad. Sci. USA, 78:2072 (1981)); neo, which confers resistance to the aminoglycoside G418 (Clinical Pharmacy, 12:488-505; Wu et al., Biotherapy, 3:87-95 (1991); Tolstoshev, Ann. Rev. Pharmacol. Toxicol., 32:573-596 (1993); Mulligan, Science, 260:926-932 (1993); Anderson, Ann. Rev. Biochem., 62:191-121 (1993); TIB TECH, 11(5):155-215 (May 1993); and hygro, which confers resistance to hygromycin (Santerre et al., Gene, 30:147 (1984)). Methods commonly known in the art of recombinant DNA technology can be routinely applied to select the desired recombinant cell clones, and such methods are described, for example, in Ausubel et al., eds., Current Protocols in Molecular Biology, John Wiley & Sons, NY (1993); Kriegler, Gene Transfer and Expression, A Laboratory Manual, Stockton Press, NY (1990); in Chapters 12 and 13, Dracopoli et al., eds., Current Protocols in Human Genetics, John Wiley & Sons, NY (1994); Colberre-Garapin et al., J. Mol. Biol., 150:1 (1981), which are incorporated by reference herein in their entireties.

In addition, the expression levels of an expressed protein molecule can be increased by vector amplification (for a review, see Bebbington, C. R. et al., Chapter 8: “The use of vectors based on gene amplification for the expression of cloned genes in mammalian cells”, Glover, D. M., ed., DNA Cloning, Vol. 3: A Practical Approach, pp. 163-188, IRL Press Limited, publ. (1987)). When a marker in the vector system expressing a protein is amplifiable, an increase in the level of inhibitor present in the host cell culture will increase the number of copies of the marker gene. Since the amplified region is associated with the protein-encoding gene, production of the protein will concomitantly increase (Crouse et al., Mol. Cell. Biol., 3:257 (1983)).

Vectors which harbor glutamine synthase (GS) or dihydrofolate reductase (DHFR) encoding nucleic acid as the selectable markers can be amplified in the presence of the drugs methionine sulphoximine or methotrexate, respectively. An advantage of glutamine synthase based vectors is the availability of cell lines (e.g., the murine myeloma cell line, NSO) which are glutamine synthase negative. Glutamine synthase expression systems can also function in glutamine synthase expressing cells (e.g., CHO cells) by providing additional inhibitor to prevent the functioning of the endogenous gene.

Vectors that express DHFR as the selectable marker include, but are not limited to, the pSV2-dhfr plasmid (Subramani et al., Mol. Cell. Biol. 1:854 (1981)). Vectors that express glutamine synthase as the selectable marker include, but are not limited to, the pEE6 expression vector described in Stephens et al., Nucl. Acids. Res., 17:7110 (1989). A glutamine synthase expression system and components thereof are detailed in PCT publications: WO 87/04462; WO 86/05807; WO 89/01036; WO 89/10404; and WO 91/06657 which are incorporated by reference herein in their entireties. In addition, glutamine synthase expression vectors that can be used in accordance with the present invention are commercially available from suppliers, including, for example, Lonza Biologics, Inc. (Portsmouth, N.H.).

### Types of Cell Cultures

For the purposes of understanding, yet without limitation, it will be appreciated by the skilled practitioner that cell cultures and culturing runs for protein production can include three general types; namely, continuous culture, batch culture and fed-batch culture. In a continuous culture, for example, fresh culture medium supplement (i.e., feeding medium) is provided to the cells during the culturing period, while old culture medium is removed daily and the product is harvested, for example, daily or continuously. In continuous culture, feeding medium can be added daily and can be added continuously, i.e., as a drip or infusion. For continuous culturing, the cells can remain in culture as long as is desired, so long as the cells remain alive and the environmental and culturing conditions are maintained.

In batch culture, cells are initially cultured in medium and this medium is neither removed, replaced, nor supplemented, i.e., the cells are not “fed” with new medium, during or before the end of the culturing run. The desired product is harvested at the end of the culturing run.

For fed-batch cultures, the culturing run time is increased by supplementing the culture medium with fresh medium during the run, i.e., the cells are “fed” with new medium (“feeding medium”) during the culturing period. Fed-batch cultures can include various feeding regimens and times, for example, daily, every other day, every two days, etc., more than once per day, or less than once per day, and so on. Further, fed-batch cultures can be fed continuously with feeding medium.

The desired product is then harvested at the end of the culturing/production run. One embodiment of the present invention embraces fed-batch cell cultures in which growth factors such as insulin and/or IGF is added at a time after inoculation.

According to the present invention, cell culture can be carried out, and proteins can be produced by cells, under conditions for the large or small scale production of proteins, using culture vessels and/or culture apparatuses that are conventionally employed for animal or mammalian cell culture. As is appreciated by those having skill in the art, tissue culture dishes, T-flasks and spinner flasks are typically used on a laboratory scale. For culturing on a larger scale, (e.g., 500 L, 5000 L, and the like, for example, as described in commonly-assigned U.S. Pat. Nos. 7,541,164 and 7,332,303, and U.S. patent application Ser. No. 12/086,786, filed Dec. 19, 2006, the contents of which are incorporated by reference herein in their entirety) procedures including, but not limited to, a fluidized bed bioreactor, a hollow fiber bioreactor, roller bottle culture, or stirred tank bioreactor systems can be used. Microcarriers may or may not be used with the roller bottle or stirred tank bioreactor systems. The systems can be operated in a batch, continuous, or fed-batch mode. In addition, the culture apparatus or system may or may not be equipped with a cell separator using filters, gravity, centrifugal force, and the like.

### Phases of Cell Culture and Associated Parameters

The term “inoculation” refers to the addition of cells to starting medium to begin the culture.

The “growth phase” of a culture is the phase during which the viable cell density at any time point is higher than at any previous time point.

The “stationary phase” of a culture is the phase during which the viable cell density is approximately constant (i.e., within measuring error) over a time period of any length.

The “death phase” of a culture is the phase that comes after the growth phase or after the growth phase and the stationary phase, and during which the viable cell density at any time point is lower than at any previous time point during that phase.

In one embodiment of the invention, the culture medium is supplemented (“fed”) during the production phase to support continued protein production, particularly in an extended production phase, and to attain ample quantities of high quality protein product. Feeding can occur on a daily basis, or according to other schedules to support cell viability and protein production.

The culturing process according to the present invention may result in more viable cell survival until the end of the culturing period. Accordingly, in some embodiments, the more cells that survive, the more cells that are producing the desired product. This, in turn, results in a greater accumulated amount of the product at the end of the culturing process, with the rate of protein production by individual cells, i.e., cell specific productivity, remaining the same. Cell specific productivity or cell specific rate, as known in the art, typically refers to the specific expression rate of product produced per cell, or per measure of cell mass or volume. Cell specific productivity is measured in grams of protein produced per cell per day, for example, and can be measured according to an integral method involving the following formulae:

dP/dt=qₚ X, or

P=qₚ∫₀^{t} Xdt

where qₚ is the cell specific productivity constant; X is the number of cells or cell volume, or cell mass equivalents; and dP/dt is the rate of protein production. Thus, qₚ can be obtained from a plot of product concentration versus time integral of viable cells (∫₀^{t} Xdt “viable cell days”). According to this formula, when the amount of protein product produced is plotted against the viable cell days, the slope is equivalent to the cell specific rate. Viable cells can be determined by several measures, for example, biomass, O₂ uptake rate, lactase dehydrogenase (LDH), packed cell volume or turbidity. (e.g., U.S. Pat. No. 5,705,364 to Etcheverry, T. et al.)
Production of aCD40L Fusion Protein by the Culturing Methods of the Present Invention

In other embodiments encompassed by the present invention, the cell culture methods of the invention are utilized to produce a soluble aCD40L fusion protein, as described below.

Soluble aCD40L is an antibody polypeptide that specifically binds human CD40L. The antibody polypeptide is a domain antibody (dAbs) comprising a variable domain. Soluble aCD40L is useful in the treatment of diseases involving CD40L activation, such as autoimmune diseases. U.S. Patent Application Ser. No. 61/655,110, filed Jun. 4, 2012 describes the aCD40L dAb and is incorporated by reference herein in its entirety.

In a preferred embodiment, soluble aCD40L is produced by recombinantly engineered host cells. The soluble aCD40L protein can be recombinantly produced by CHO cells transfected with a vector containing the DNA sequence encoding soluble aCD40L. The soluble aCD40L fusion protein is produced in high quantity and when cultured in accordance with the processes of this invention. The invention affords the production of high levels of recoverable protein product, e.g., soluble aCD40L protein product as shown in Example 5.

### EXAMPLES

The following Examples set forth specific aspects of the invention to illustrate the invention and provide a description of the present methods for those of skill in the art. The Examples should not be construed as limiting the invention, as the Examples merely provide specific methodology and exemplification that are useful in the understanding and practice of the invention and its various aspects.

Examples 1-5 as set forth below describe experiments relating to cell culture processes involving the culturing process of the invention with and without growth factors.

### Example 1

### CHO Cells Were Able to Grow Under GF-Free Condition

1. Thaw a new vial of cells in early passage and culture in platform medium (with 1 or 10 mg/L insulin) for 2 passages.
2. At passage 3, transfer the cells to basal media without insulin, and keep splitting every 3 to 4 days at a density of 0.6×10⁶ cells/ml.
3. In the first few passages under insulin free conditions, cell growth may significantly slow down and viability may drop (˜80%). Meanwhile, ammonium production may be increased due to insufficient glucose uptake and oxidation of amino acids as an energy source. As a result, pH in flasks may increase. CO₂ level may need to be adjusted/increased accordingly to control pH in a range of 7.0-7.3. This is very important to maintain the cells in a healthy state.
4. In general, spent medium carried into a new passage should be less than 30%. In case cell growth is too slow to satisfy the criterion, cells could be centrifuged down and transferred at a desired number to a new flask which contains 30% spent medium and 70% fresh medium.
5. When cells are fully adapted, cell growth will recover and viability will come back to over 95%. It may take up to 7˜9 passages to adapt the cells to insulin free condition depending on the specific clone.
6. Cell culture parameters: shaking speed: 150 rpm; shake flask: 250 ml or 500 ml baffled shake flasks (Corning Inc.) with 100 ml or 200 ml working volume; temperature: 37° C.; CO₂: 6% in general, may be adjusted to control pH at 7.0˜7.3 (up to 8% CO₂)

### Example 2

### Removal of Insulin did not Drastically Impact the mTOR Pathway

The purpose of this study was to use antibody array to compare the phosphorylation/expression level of proteins involved in mTOR in cell grown with and without growth factor, insulin, thereby demonstrating that from a molecular and cell biology perspective that cells are able to grow under GF-free condition.

### Antibody Array Sample Preparation

Clone 40A6 cultured under insulin-free or insulin containing basal medium. 5×10⁶ viable cells were sampled for each condition on day 3. Cells were centrifuged down at 500 g for 5 min and at 4° C. Cells were washed with 10 ml ice cold 1× PBS, and centrifuged down at 500 g for 5 min and at 4° C. Cells were always kept on ice or 4° C. during sample processing. After dumping the PBS, cells were frozen at −70° C. immediately and stored at −70° C. till antibody array analysis

### Antibody Array Protocol

### Protein Extraction

Wash the cells with ice cold 1× PBS. Add Lysis Beads and Extraction Buffer to the sample. Mix rigorously by vortexing for 30 seconds. Incubate the mixture on ice for 10 minutes. Repeat vortexing for 30 seconds at 10-minute intervals for 60 minutes. Incubate the mixture on ice between vortexing. Centrifuge the mixture at 10,000×g for 20 minutes at 4° C. Transfer the supernatant to a clean tube. Use spin columns to change the buffer in the supernatant to Labeling Buffer. Measure protein concentration. Note: phosphatase inhibitors are included in Extraction Buffer and Labeling Buffer.

### Protein Labeling

Add 100 μL of DMF to 1 mg of Biotin Reagent to give a final concentration of 10 μg/μL. Add Labeling Buffer to the protein sample to bring the volume to 75 μL. Add 3 μL of the Biotin/DMF to the protein sample with Labeling Buffer. Mix and incubate at room temperature for two hours with mixing. Add 35 μL of Stop Reagent. Incubate for 30 minutes at room temperature with mixing.

### Coupling

Blocking: Submerge Antibody Microarray in Blocking Buffer. Shake for 40 minutes at room temperature. Rinse the slide with MILLI-Q® grade water.

Incubate the slide in Coupling Chamber with 85 μg of labeled protein sample in 6 mL Coupling Solution on an orbital shaker for 2 hours at room temperature. Remove the slide from the coupling chamber. Wash the slide three times with fresh Wash Buffer. Rinse extensively with DI water.

### Detection

Add 30 μl of Cy3-Streptavidin (1 mg/ml) to the 60-ml bottle containing Detection Buffer. Submerge the slide in 30 ml of Cy3-Streptavidin solution. Incubate on an orbital shaker for 45 minutes at room temperature in the dark. Wash the slide three times with fresh Wash Buffer. Rinse extensively with DI water. Dry the slide with compressed nitrogen. Scan on Axon GenePix Array Scanner.

### Assay Data

For each spot on the array, median spot intensity is extracted from array image. Using the median intensity, determine the average signal of the replicate spots for each antibody. The data is labeled as Average Signal of Replicate Spots on the Array. The CV of the Replicates on the Array is the coefficient of variation of the replicate spots for each antibody. For normalization, within each array slide the mean value of the Average Signal of all antibodies in the array is determined. This value is presented as Mean Signal.

Normalized data=Average Signal of Replicate Spots/Mean Signal

Using the normalized data, determine the fold change between control and treated samples.

Signal Fold change=Treated/Control

### Example 3

### GFs/Proteins/Peptides Free Media Improve Process Performance

Cells: Clone 63C2 producing aCD40L fusion protein.

### Cell Culture Parameters:

Shaking speed: 150 rpm; shake flask: 250 ml or 500 ml baffled shake flasks (Corning Inc.) with 100 ml or 200 ml working volume; temperature: 37° C.; CO₂: 6% in general.

Basal: medium with or without insulin.

Feed: medium with or without insulin and LONG®R3.

INS-Free: no insulin in basal, no insulin and no LONG®R3 in feed.

INS: 1 mg/L insulin in basal, 10 mg/L insulin in feed.

Feeding strategy: feeding begins on day 3, feed 3.64% initial culture volume till day 14.

Sampling: at designated time points, samples were taken to measure cell density and viability with CEDEX®, titer with HPLC, and high molecular species with size exclusion chromatography (SE).

### Example 4

### GF Add-Back to GF-Free Condition Boosted Cell Growth and Protein Production

Cells: Clone 63C2 producing aCD40L fusion protein.

### Cell Culture Parameters:

Agitation: 120 rpm; 5 L reactor with 1.3 L initial culture volume; temperature: 37° C.; dissolved oxygen (DO) controlled at 50%.

Basal: medium with or without insulin.

Feed: with or without insulin and LONG®R3.

INS-Free: no insulin in basal, no insulin and no LONG®R3 in feed.

INS: 1 mg/L insulin in basal, 10 mg/L insulin in feed.

INS Add-back: 1 mg/L insulin and 10 mg/L insulin were added back to basal and feed, respectively. Inoculum was insulin-free cells.

Feeding strategy: feeding begins on day 3, feed 3.64% initial culture volume till day 14.

Sampling: at designated time points, samples were taken to measure cell density and viability with CEDEX®, titer with HPLC, and high molecular species with size exclusion chromatography (SE)

### Example 5

### GFs/Proteins/Peptides Free Media Improve Process Performance

Cells: Clone 63C2 producing aCD40L fusion protein.

### Cell Culture Parameters:

Agitation: 120 rpm; 5 L reactor with 1.3 L initial culture volume; temperature: 37° C.; dissolved oxygen (DO) controlled at 50%.

Basal: with or without insulin.

Feed: with or without insulin and LONG®R3.

INS-Free+LR3: 4 μg/L LR3 and 40 μg/L LR3 were added to basal feed medium, respectively. Inoculum was GF-free.

INS+LR3: 4 μg/L LR3 and 40 μg/L LR3 were added to basal with 1 mg/L and feed medium with 10 mg/L insulin, respectively. Inoculum was in basal with 1 mg/L insulin.

Feeding strategy: feeding begins on day 3, feed 3.64% initial culture volume till day 14.

Sampling: at designated time points, samples were taken to measure cell density and viability with CEDEX®, titer with HPLC, and high molecular species with size exclusion chromatography (SE).

What is claimed is:

1. A cell culture process for the production of proteins, comprising:
a) culturing CHO cells in growth factor, protein and peptide free media in cell culture conditions that allow for protein production; and
b) feeding the cells with one or more growth factors.

2. The cell culture process of claim 1, wherein the feeding step b) occurs on a daily basis.

3. The cell culture process of claim 1, wherein the feeding step b) occurs on a continuous basis.

4. The cell culture process of claim 1, wherein the feeding step b) occurs more than once per day.

5. The cell culture process of claim 1, wherein the feeding step b) occurs less than once per day.

6. The cell culture process of claim 1, wherein the growth factor is added to the basal media, the feed media or as a bolus anytime after inoculation.

7. The cell culture process of claim 6, wherein the growth factor is fed to the cells in feeding medium.

8. The cell culture process of claims 1-7, wherein the growth factor is selected from the group consisting of insulin, epidermal growth factor (EGF), insulin-like growth factor (IGF), transforming growth factor alpha (TGF-α), bFGF and PDGF.

9. The cell culture process of claim 7, wherein the growth factor is insulin and the feeding medium contains insulin in an amount of about 1 μg/L to 10 mg/L.

10. The cell culture process of claim 6, wherein the growth factor is insulin and the insulin level is maintained in the cell culture at a concentration 1 μg/L to 10 mg/L.

11. The cell culture process of claim 1, wherein end stage cell viability is increased compared to end stage cell viability in cells grown in media comprising growth factor.

12. The cell culture process of claim 1, wherein protein titer is increased compared to protein titer in cells grown in media comprising growth factor.

13. The cell culture process of claim 1, wherein protein aggregation is decreased compared to protein aggregation in cells grown in media comprising growth factor.

14. A cell culture process for the production of a soluble aCD40L fusion protein, comprising:
a) culturing CHO cells in growth factor, protein and peptide free media in cell culture conditions that allow for aCD40L production; and
b) feeding the cells with one or more growth factors.

15. A cell culture process for the production of proteins, comprising: culturing CHO cells in growth factor, protein and peptide free media under cell culture conditions that allow for protein production.

16. The cell culture process of claim 15, wherein end stage cell viability is increased compared to end stage cell viability in cells grown in media comprising growth factor.

17. The cell culture process of claim 15, wherein protein titer is increased compared to protein titer in cells grown in media comprising growth factor.

18. The cell culture process of claim 15, wherein protein aggregation is decreased compared to protein aggregation in cells grown in media comprising growth factor.
